(19) United States
(12) Patent Application Publication
(10) Pub. No.: US 2007/0292508 A1
(43) Pub. Date: Dec. 20, 2007

(54) ORALLY DISINTEGRATING DOSAGE FORMS

(75) Inventors: Janos Szamosi, Greentown, PA (US); Liangping Yu, Mahwah, NJ (US); Paul H. Richardson, Vernon, NJ (US)

Correspondence Address: HOFFMANN & BARON, LLP, 6900 JERICHO TURNPIKE, SYOSSET, NY, 11791, US

(73) Assignee: Balchem Corporation, New Hampton, NY (US)

(21) Appl. No.: 11/754,507

(22) Filed: May 29, 2007

Related U.S. Application Data
(60) Provisional application No. 60/811,057, filed on Jun. 5, 2006.

(51) Int. Cl. A61K 35/74; A61K 31/522; A61K 36/18; A61K 31/19; A61K 31/16; A61K 9/26

(52) U.S. Cl. 424/469; 514/569; 514/629; 424/725; 424/93.45; 514/282; 514/263.31

(57) ABSTRACT

The present invention is directed to orally disintegrating dosage forms comprising lipid coated substrates and silicified excipients. The use of silicified excipients in the orally disintegrating dosage form along with lipid coating of active agents, allows for improvements in the ability to prepare these dosage forms. Further, the dosage form can prevent unpleasant taste or aftertaste and provide better chemical and mechanical stability of the coated active substrate. This present invention also provides the possibility of harder more durable tablets, along with targeted immediate or modified release profiles for the active agent.

### CROSS-REFERENCE TO RELATED APPLICATION

This application claims the benefit of U.S. Provisional Application No. 60/811,057, Filed Jun. 5, 2006, which is incorporated herein by reference.

### FIELD OF THE INVENTION

The present invention is directed to orally disintegrating dosage forms comprising lipid coated substrates and silicified excipients. In particular the silicified excipient is silicified microcrystalline cellulose. Use of silicified microcrystalline cellulose in the orally disintegrating dosage from along with lipid coating of active agents, allows for improvements in the ability of the protective coating to prevent unpleasant taste or aftertaste and provide better chemical and mechanical stability of the coated substrate. Further, immediate and modified release profiles are possible. This formulation also provides a practical method of preparing durable orally disintegrating dosage forms.

### BACKGROUND OF THE INVENTION

Oral administration is the preferred route for numerous pharmaceuticals and nutraceuticals. Orally disintegrating dosage forms offer advantages in terms of convenience and ease of use. However, for many actives, problems such as instability, rapid degradation

To overcome many of the problems listed above, a variety of methods have been used in attempts to effectively protect active agents by controlling release rates and/or masking unpleasant tastes or aftertastes. One common method is to coat the active agent with layers of various polymeric coatings. An alternative approach involves coating the active agent with layers of hydrophobic materials such as lipids or waxes.

Although many orally disintegrating dosage forms exist, some of which use approaches where active substrates are coated, these dosage forms often do not provide a satisfactory level of taste masking within a durable dosage form. There exists a need to optimize both the type of active agent coating along with the dosage form excipients to maximize the level of taste masking, release characteristics and dosage form physical properties such as disintegration and tablet durability.

### SUMMARY OF THE INVENTION

It is an object of the invention to provide an orally disintegrating dosage form that comprises a plurality of lipid coated active substrates, and a silicified excipient. This combination results in an orally disintegrating dosage form that has excellent taste masking properties as well as providing a commercially viable dosage form.

It is an object of the invention to provide a method of preparing an orally disintegrating dosage form that comprises a plurality of lipid coated active substrates, and a silicified excipient. and unpleasant taste or aftertaste can make oral administration less than favorable.

It is an object of the invention to provide an oral disintegrating dosage form that comprise a plurality of lipid coated active substrates, and a silicified excipient, where the coated active substrate is taste masked and has immediate release.

It is an object of the invention to provide an oral disintegrating dosage form that comprise a plurality of lipid coated active substrates, and a silicified excipient, where the coated active substrate is taste masked and has modified release.

It is further an object of the invention to provide an oral disintegrating dosage form that comprises a plurality of lipid coated active substrates, and a silicified excipient, where the combination of lipid coated active substrates and silicified excipient contributes to the mechanical strength and stability of the dosage form, and the chemical and physical stability of the active ingredient.

In accordance with the above objects and others, the present invention is directed in part to a method for preparing lipid coated active substrates comprising an active agent. In certain embodiments, the method comprises melting a lipid to a temperature above its melting point to obtain a coating solution and then applying the coating solution to the substrates at a temperature such that the coating solidifies to form uniformly coated substrates. In certain embodiments, the coating may be applied to be substrates via various spraying techniques, e.g., utilizing a fluidized bed type encapsulation process.

The substrates of the present invention may comprise a pharmaceutically acceptable bead, granule, spheroid, pellet, slab, rod and the like.

In certain embodiments, the substrates may comprise a mixture of an active agent and an excipient.

In order that the invention describe herein may be further understood, the following definitions are provided for the purposes of the disclosure:

The term “oral disintegrating dosage form” is defined as a tablet, caplet, rod, spheroid, film strip or any other dosage form that disintegrates/disperses in the buccal cavity with the help of saliva, i.e., without the need of additional water. The time of disintegration should be less than two minutes, preferably less than one minute, and even more preferably less than 30 seconds.

The term “active agent” or “active ingredient” is defined as any compound that provides an effect in an environment of use. In certain embodiments, the effect is a therapeutic effect. An active agent may be active pharmaceutical ingredient (API) such as a drug or biological agent, a nutraceutical agent, an herbal remedy, a vaccine, or a bactericidal agent.

The term “excipient” is defined as any pharmaceutically acceptable excipient suitable for animal, e.g., human, consumption.

The term “substrate” is defined as the active agent itself, the active agent combined together with at least one pharmaceutically acceptable excipient. The substrate may be in the form of a pharmaceutically acceptably bead, granule, pellet, spheroid, and the like with the active agent contained therein or thereon.

For purposes of the present invention, the term “patient” is defined as a human or animal inflicted with a disease or condition that requires treatment with a pharmaceutically active agent.

The term “object” is defined as a human or animal that does not have any disease or condition that requires treatment with a pharmaceutically active agent, e.g., a normal volunteer.

The term “protective coating” is defined as a coating that may have a positive effect on the processability of the substrate (e.g., taste-making, improvement in flow, binding, friability, hardness, etc).

The term “lipid” is defined as any lipid known in the art, including fatty acid glycerol esters, hydrogenated vegetable oils or animal fats, waxes, fatty acids, fatty alcohols, sterols, and phospholipids, or combinations thereof.

The term “hydrophobic material” is defined as material that is acceptable for the active defined herein, that has a sufficient level of hydrophobicity that is dissolvable in a lipid.

The term “polymer” is defined as a chemical with greater than 5 monomer units.

There term “silicified excipient” is defined as any inactive or active that is combined or impregnated with silicon, silica, silicates or its derivatives.

For purposes of the present invention, the term “immediate release” is defined as a release of substantially all of the active agent contained in a dosage form within about 1 hour (and preferably faster)after being exposed to an environment of use (e.g., an in-vitro dissolution bath, or the stomach of a human patient).

For purposes of the present invention, the term “modified release” is defined as delayed, controlled, extended, site specific, slow or pulsatile release, i.e., different from immediate release, of the active agent from a dosage form.

### DETAILED DESCRIPTION

The combination of a coated substrate with a silicified excipient in an orally disintegrating dosage form, where the coated substrate is coated with a lipid protective coating, provides improved properties. These improved properties include preventing unpleasant taste or after taste and contribute to the mechanical strength, chemical protection and stability of the coated substrate, the to the processability of the dosage form and to a modified release of the active agent. In addition, the silicified excipient aids in the preparation of a pre-mix that is subsequently tableted, as well as providing an orally disintegrating dosage form that has favorable mechanical properties.

It is well known that a coated substrate, where the protective coating is lipid, can mask taste. However, the taste masking and level of protection of the protective coating is compromised owing to mechanical pressures, when it is included in an orally disintegrating dosage form. In the present invention, by using a silicified excipient as part of the orally disintegrating dosage form, the lipid coated substrate retains beneficial properties.

It has been found that upon blending lipid coated substrates with conventional flow aids such as silica in a tablet blend formulation, the efficiency of sieving the blend is severely compromised. This leads to long times to sieve which can be costly in a manufacturing environment. Conversely, by using a co-processed silicified excipient, with lipid coated substrates, the sieving process is very efficient.

The formulations of the present invention allow for a superior performance orally disintegrating dosage form, both in terms of ease and time of manufacture, and final properties such as taste masking, stability, release and physical properties of the dosage form.

Protective Coating—Microencapsulation

The protective coating is lipid, composed of a lipid with optionally other coating additives. Not limiting to this property, any lipid that exhibits acceptable properties for use in hot-melt processes can be used in the protective coatings of the present invention. Lipids suitable for use as a protective coating in the oral disintegrating dosage forms of the invention are well known to those skilled in the art, and basically are fat and fat-like substances which are derived from plants, animals or synthetically. A variety of materials having varied chemical structure are generally classified as lipids, and are considered to be useful in the formulations and methods of the present invention.

One type of lipid suitable for use in the invention is generally classified as fatty acid glycerol esters. Such materials, also known as glycerides, and may be simple (where all fatty acid groups are identical) or mixed, saturated or unsaturated. Examples of suitable glycerides include those derived from higher-molecular weight fatty (aliphatic) acids, such as palmitic, stearic and oleic acids. For example, the lipid may be a fatty acid glycerol ester, such as, but not limited to, mono-,di-, tri-glycerides and any combinations or mixtures thereof. Another type of lipid suitable suitable for use in the invention is generally classified as waxes, which are esters of high molecular weight, even-numbered monohydric alcohols (C₁₆ to C₃₆) and fatty acids (C₁₄ to C₃₆). Further detail and explanation concerning acceptable lipids useful in the invention may be gleaned from Remington's 20^{th} Edition ©2000 by the University of the Sciences in Philadelphia, pages 415-419, hereby incorporated by reference.

In particular, hydrogenated vegetables oils and animal oils may be used in the protective coating in the present invention. Hydrogenated vegetable oils may include, but are not limited to, cashew, castor bean, linseed, grape seed, hemp seed, mustard seed, poppy seed, rape seed (canola oil), safflower, sesame seed, sunflower, almond, algae, apricot, argan, avacodo, corn oil, cotton seed, coconut, fusarium, hazelnut, neem oil, palm, palm kernel, peanut, pumpkin, rice bran, walnut, soybean oil and any combinations or mixtures thereof.

The protective coating may also be comprised of a wax such as, but not limited to, paraffin wax; a petroleum wax; a mineral wax such as ozokerite, ceresin, utah wax or montan wax; a vegetable wax such as, for example, carnauba wax, japan wax, bayberry wax or flax wax; an animal wax such as, for example, spermaceti; or an insect wax such as beeswax, Chinese wax or shellac wax.

The choice of lipid for preparing the protective coatings of the present invention may have varying effects on the methods of preparation as well on the dissolution of the active agent from the coated substrate.

The amount of protective coating is at least about 1% to not more than 90% by weight of the microencapsulated substrate. In certain embodiments, the coating is at least about 1% to about 60% by weight of the microencapsulated substrate, preferably at least about 1% to 35% by weight, and even more preferably at least about 1% to about 20% by weight, and even more preferably at least about 1% to about 10% by weight of the microencapsulated substrate.

Coating additives can be included or dissolved in the lipid. Optional additives for use in the present invention include, but are not limited to, flavoring agents, taste-making agents, bitter blockers, plasticizers, binders, sensory masking agents, flavors, pH triggers, antioxidants, cellulose and cellulose derivatives, and the like. Other excipients suitable for use in the present invention are described in the Handbook of Pharmaceutical Excipients, American Pharmaceutical Association (2005), incorporated by reference herein.

The protective coating may comprise from about 0 to about 25 percent by weight coating additives (in addition to the lipid and the optional hydrophobic material), and preferably such additives comprise up to about 10 percent by weight of the protective coating.

In certain embodiments of the invention, an effective amount of a hydrophobic material is incorporated into the protective coating. The hydrophobic material, preferably a cellulose derivative (e.g., ethylcellulose), is dispersed or (preferably) dissolved in the lipid prior to coating may range from about 1% to about 25%, from about 1% to about 15% or from about 1% to about 10%. The addition of cellulose derivative(s) to the protective coating may improve the tableting properties of the coated substrate. Improved tableting properties include, but are not limited to increased mechanical strength stability, binding, friability and hardness of the dosage form, as well as providing consistent properties from tablet to tablet, as described in Applicants' co-pending Provisional Patent Application No. 60/810,983, entitled “Protective Coating for Active Agent Substrate”, filed Jun. 5, 2006, disclosure of which is hereby incorporated by reference.

The protective coating is applied by any known coating method. These include but are not limited to spray chilling, spinning disk, pan coating, fluidized bed coating. In particular fluidized bed type microencapsulation processes known in the art may be utilized such as those described in U.S. Pat. Nos. 4,511,584; 4,537,784; 4,511,592; and 4,497,845, the disclosures of which are hereby incorporated by reference. plurality of substrates may be delivered into a chamber (fluidized bed) and the protective coating uniformly sprayed onto the substrates. It is contemplated that temperatures inside the fluidized bed will be less than about 150° F., but not limited to. In certain embodiments, the temperature may be less than 120° F. or less than 100° F. The temperatures utilized inside the fluidized bed will be dependent upon the crystallization properties of the particular lipid to be applied, with relatively higher temperatures being preferred where the melting point of the particular lipid material utilized is relatively higher.

Fluidized-bed apparatuses suitable for microencapsulating the substrates in accordance with the methods of the present invention may be any known fluidized bed apparatus such as, but not limited to, the GPCG series of batch fluidized bed apparatus, the GF series of continuous fluidized bed apparatuses and the ProCell series of spouted bed systems manufactured by the Glatt® Group.

Preferably, the protective coating is applied in a single step process and in the absence of any required solvents.

The potential functions of the protective coatings of the present invention include, but are not limited to, high dissolution of the substrate, modified release characteristics of the substrate, enhancing the bioavailability of the active agent, improving flow, compactibility, etc. for the processing of solid dosage forms.

Substrates

The substrate to be microencapsulated may be the active agent itself, the active agent combined together with one or more acceptable excipients into suitably sized particles (granules), shaped into pellets, or manufactured as spheroids. In certain embodiments, the active agent itself is a granulate of acceptable size such that the protective coating can be directly applied onto its surface in an even manner to create a desirable microencapsulate. In other embodiments of the invention, the active agent is granulated (e.g., wet granulated) together with an excipient(s) to make desirable granules which can be coated. In such embodiments, the active agent is typically wet granulated with a diluent (e.g., lactose, sucrose, starch, and the like). Generally, the resultant granulated has a particle size ranging from about 0.01 mm to about 3 mm, and preferably from about 0.1 mm to about 1 mm. In certain preferred embodiments, the active agent granulate is about 800 microns to about 200 microns in diameter (and in certain embodiments about 400 microns) is then separated and further processed via microencapsulation. Alternatively, the substrates used in the invention may comprise a pharmaceutically acceptable sugar sphere (bead) coated with the active agent. Sugar spheres are solid excipients which are composed of one or more sugar, starch, cellulose, etc. and typically have a size ranging about 300 microns to about 1400 microns. Pellets are generally considered in the art to comprise small, sterile cylinders (e.g., about 3 mm in diameter by about 8 mm in length), which are formed from compression from a mass comprising active agent and one or more excipients. On the other hand, the substrate may comprise a matrix spheroid in which the active agent is incorporated together with the excipient(s) a substantially uniform fashion. One skilled in the art will also appreciate that excipients may be utilized in the preparation of such substrates without changing the basic character of the invention.

The load of the active agent contained in the coated substrates may be at least about 10%. In certain embodiments, the load may be at least about 40%. In certain embodiments, the load may be at least 65%. In certain embodiments, the load may be at least about 75%. In certain embodiments, the load may be at least 95%. When the present invention contemplates lipid coated acetaminophen substrates, the load of acetaminophen ranges from about 85% to about 97%.

Active Agents

Active agents suitable for use in the present invention may include, but are not limited to, water soluble and water insoluble agents. Active agents include drugs, nutrients, biologicals, vaccines and herbal agents.

Combinations of active agents may be included within the dosage form. In this manner, the active agents can be combined within the coated active substrate and/or included with the excipients of the dosage form.

Drugs

Examples of active agents that are suitable for incorporation in the present invention include: antihistamines (e.g., azatadine maleate, brompheniramine maleate, carbinoxamine maleate, chlorpheniramine maleate, dexchlorpheniramine maleate, diphenhydramine hydrochloride, doxylamine succinate, methdilazine hydrochloride, promethazine, trimeprazine tartrate, tripelennamine citrate, tripelennamine hydrochloride and triprolidine hydrochloride);antibiotics (e.g., penicillin V potassium, cloxacillin sodium,dicloxacillin sodium, nafacillin sodium, oxacillin sodium, carbenicillin indanyl sodium, oxytetracycline hydrochloride, tetracycline hydrochloride, clinamycin phosphate, clindamycin hydrochloride, clindamycin palmitate HCL, lincomycin HCL, novobiocin sodium, nitrofurantonin sodium, metronodazle hydrochloride); antituberculosis agents (e.g., isoniazed); cholinergic agents(e.g., ambenonium chloride, bethanecol methylbromide, clindinium bromide, dicyclomine hydrochloride,glyopyrrolate, hexocyclium methylsulfate, homatropine methylbromide, hyoscyamine sulfate, methantheline bromide, hyoscine hydrobromide, oxyphenonium bromide, propantheline bromide, tridihexethyl chloride); sympathomimetics (e.g., bitolterol mesylate, ephedrine, ephedrine hydrochloride, ephedrine sulphate, orciprenaline sulphate, phenylpropanolamine hydrochloride, pseudoephedrine hydrochloride, ritodrine hydrochloride, salbutamol sulphate, terbutaline sulphate); sympatholytic agents (e.g., phenoxybenzamine hydrochloride); miscellaneous autonomic drugs (e.g., nicotine); iron preperations (e.g., ferrous gluconate, ferrous sulphate); haemostatics (e.g., aminocaproic acid); cardiac drugs (e.g., acebutolol hydrochloride, disopyramide phosphate, flecainide acetate, procainamide hydrochloride, propranolol hydrochloride, quinidine gluconate, timolol maleate, tocainide hydrochloride, verapamil hydrochloride); antihypertensive agents (e.g., captopril, clonidine hydrochloride, hydralazine hydrochloride, mecamylamine hydrochloride, metoprolol tartrate); vasodilators (e.g., papaverine hydrochloride);non-steroidal anti-inflammatory agents (e.g., choline salicylate, ibuprofren , ketoprofen, magnesium salicylate, meclofenamate sodium, naproxen sodium, tolmetin sodium); opiate agonists (e.g., codeine hydrochloride, codeine phosphate, codeine sulphate, dextromoramide tartrate, hydrocodone bitartrate, hydromorphone hydrochloride, pethidine hydrochloride, methadone hydrochloride, morphine sulphate, morphine acetate, morphine lactate, morphine meconate, morphine nitrate, morphine monobasic phosphate, morphine tartrate, morphine valerate, morphine hydrobromide, morphine hydrochloride, propoxyphene hydrochloride); anticonvulsants (e.g., phenobarbital sodium, phenytoin sodium, troxidone, ethosuximide, valproate sodium); tranquilizers (e.g., acetophenazine maleate, chlorpromazine hydrochloride, fluphenazine hydrochloride, prochlorperazine edisylate, promethazine hydrochloride, thioridazine hydrochloride, trifluoroperazine hydrochloride, lithium citrate, molindone hydrochloride, thiothixine hydrochloride); chemotherapeutic agents (e.g., doxorubicin, cisplatin, floxuridine, methotrexate, combinations thereof, etc); lipid lowering agents (e.g., gemfibrozil, clofibrate, HMG-CoA reductase inhibitors, such as for example, atorastatin, cerivastatin, fluvastatin, lovastatin, pravastatin, simvstatin, etc.); H₂-antagonists (e.g., cimetidine, famotidine, nizatidine, ranidine HCl, etc); anti-coagulant and anti-platelet agents (e.g., warfarin, cipyridamole, ticlopidine,etc.); bronchodilators (e.g., albuterol, isoproterenol, metaproterenol, terbutaline, etc.); stimulants (e.g., benzamphetamine hydrochloride, dextroamphetamine sulphate, dextroamphetamine phosphate, diethylpropion hydrochloride, fenfluramine sulphate, methamphetamine hydrochloride, methylphenidate hydrochloride, phendimetrazine tartrate, phenmetrazine hydrochloride, caffeine citrate); barbituates (e.g., amylobarbital sodium, butabarbital sodium, secoarbital sodium);sedatives (e.g., hydroxydize hydrochloride, methprylon); expectorants (e.g., potassium iodide); antiemetics (e.g., benzaquinamide hydrochloride, metoclopropamide hydrochloride, trimethobenzamide hydrochloride); gastro-intestinal drugs (e.g., ranitidine hydrochloride); heavy metal antagonists (e.g., penicillamine, penicillimine hydrochloride); antithyroid agents (e.g., methimazole); genitourinary smooth muscle relaxants (e.g., flavoxate hydrochloride, oxybutynin hydrochloride); vitamins (e.g., thiamine hydrochloride, ascorbic acid); unclassified agents (e.g., amantadine hydrochloride, colchicine, etidronate disodium, leucovorin calcium, methylene blue, potassium chloride, pralidoxime chloride; steroids, particularly glucocorticoids (e.g., prednisolone, prednisone, cortisone, hydrocortisone, methylprednisolone, betamethasone, dexamethasone, triamcinolone), and any combinations or mixtures of the foregoing.

In other embodiments, the subtrates may comprise a combination of active agents. For example, the substrates may comprise a combination of acetaminophen and an opoid analgesic, such as but not limited to, alfentanil, allylprodine, alphaprodine, anileridine, bezylmorphine, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene ethylmorphine, etonitazene, fentanyl,heroin,hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, mixtures thereof and pharmaceutically acceptable salts thereof.

In certain embodiments, the active agent is ranitidine HCl, caffeine, vitamin C, acetaminophen, or guaifenesin.

Biologicals and Vaccines

Biologicals suitable for use in the present invention include, but are not limited to, immune serums (e.g., immune globulins), antitoxins, antivenins, toxoids (e.g., tetanus toxoid), allergenic extracts, in-vivo diagnostic biologicals, interferon and the like.

Suitable vaccines include, but are not limited to, bacterial vaccines and viral vaccines. Bacterial vaccines include, but are not limited to, BCG vaccine, mixed respiratory vaccines, meningococcal vaccine, cholera vaccine, plague vaccine, pneumococcal vaccine, hemophilus vaccine and the like. Viral vaccines include, but are not limited to, measles, mumps, rubella, poliovirus, influenza, encephalitis, yellow fever, hepatitis A, hepatitus B, varicella vaccines and the like.

Herbal Agents

Herbal compositions may include, but are not limited to agrimony, alfalfa, allspice, angelica, anise, basil, bayberry, boneset, borage, caraway, cayenne, chamomile, dandelion, dill, Echinacea, evening primrose, fennel, garlic, ginger. ginko balboa, jasmine, juniper, lavendar, lemon balm, rosemary, rue, thyme, valerian, yarrow and any other herbal that is suitable for administration to a subject/patient in need thereof. Other herbal agents suitable for use in the present invention include, but are not limited to thos described in The Complete Guide to Herbal Medicines, Fetrow, Charles A., et al. (September 2000), the disclosure of which is hereby incorporated by reference.

Nutrients

Other ingredients which may be employed as the active agent(s) in the present invention include nutritional supplements, dietary supplements and combinations thereof. The compounds meeting this criteria may have varying degrees of solubility in water ranging from highly soluble to insoluble. These compounds generally include vitamins, minerals, amino acids, herbal and botanical products and the like. Vitamins generally refer to organic substances that are required in the diet and include thiamin, riboflavin, nicotinic acid, pantothenic acid, pyrodoxine, biotin, folic acid, vitamin B12, as well as enzymes and coenzymes thereof. Minerals include inorganic substances which are required in the human diet and include calcium, iron, zinc, selenium, copper, iodine, magnesium, phosphorous, chromium, and the like and mixtures thereof.

Dietary supplements which may employed as the active agent(s) of the invention include, for example, B pollen, bran, wheat germ, kelp, cod liver oil, ginseng, fish oils, amino acids, protein and the like and mixtures thereof. A non-limiting example of a final formulation comprising multi-vitamin and mineral supplements is described e.g., in U.S. Pat. 6,987,098, hereby incorporated by reference. The supplement described therein is useful for human consumption and comprises, e.g., from about 5000 I,U to about 10,000 I.U. of vitamin A; from about 1000 mg to about 2000 mg of vitamin C; about 800 I.U. of vitamin D; from about 800 I.U. to about 1200 I.U. of vitamin E; about 25 mcg of vitamin K; about 3 mg of vitamin B6; about 800 mcg of folic acid; about 400 mcg of vitamin B12; about 300 mcg of biotin; about 10 mg of pantothenic acid; up to about 18 mg of iron dosed in the form of an acceptable iodine compound; about 150 mcg of iodine dosed in the form of an acceptable zinc compound; from about 100 mcg to 200 mcg of selenium; about 2 mg of copper dosed in the form of an acceptable copper compound; about 100 mcg of chromium dosed in the form of an acceptable chromium compound; about 400 mg of potassium dosed in the form of an acceptable potassium compound; about 500 mg of choline dosed in the form of an acceptable choline compound; about 10 mg of lycopene; and about 50 mg co-enzyme Q-10 dosed in the form of an acceptable co-enzyme Q-10 compound.

Preparation of Final Dosage Form

The lipid coated active substrates may be incorporated into an orally disintegrating dosage form. The dosage form preferably disintegrates/disperses in the buccal cavity with the help of saliva, i.e., without the need of additional water, generating for more readily swallowable residual. The time of disintegration should be less than two minutes, preferably less than one minute, and even more preferably less than 30 seconds.

There are a number of technologies that generate tablets which satisfy the above requirements: Zydis® (Cardinal Health) and Quicksolv® (Janssen Pharmaceutica) use lyophilization; OraSov® (Cima Labs), Wowtab® (Yamanouchi), Flashtab® (Ethypharm) and Frosta® (Akins) are made on regular tablet presses; while FlashDose® (Biovail) uses the so-called Fuisz technology, also known as the “cotton candy process.” Examples of the actice pharmaceutical ingredients that have been commercially available in rapid-dissolve tablet form are lortadine, acetaminophen, fluoxetine, diphenydramine, famotidine, etc.

In certain embodiments, the lipid coated active substrates utilized in the present invention may be directly compressed together with or without additional pharmaceutical excipients, as described in Applicants' co-pending Provisional Patent Application No. 60/811,056, entitled “Directly Compressed Dosage Forms and Methods for Producing the Same”, filed Jun. 5, 2006, the disclosure of which is hereby incorporated by reference.

In certain embodiments, the lipid coated substrates are directly compressed together with a silicified excipient, such as ProSolv R (silicified microcrystalline cellulose). ProSolv® is a high functionally ingredient as it is multifunctional, requires less complex processing (direct compression), has high inherent functionality and imparts that functionality to the drug formula. Various grades of ProSolv® (98% microcrystalline cellulose and 2% colloidal silicon dioxide) are available from JRS Pharma Inc., Petterson, N.Y. ProSolv SMCC® 50, ProSolv SMCC® 90 and SMCC® HD (high density) 90. ProSolv SMCC® 50 has a median particle size (by sieve analysis) in the region of 50 μm while ProSolv SMCC® 90 and SMCC® HD 90 have median particle size (by sieve analysis) in the region of 90 μm. ProSolv® and the process for its manufacture are protected by U.S. Pat. Nos. 5,585,115; 5,725,884; 6,103,219; 6,217,909; 6,358,533; 6,521,261; 6,858,231; 5,725,883; 5,866,166; 6,106,865; 6,936,277; 5,741,524; 5,858,412, the disclosures of which are hereby incorporated by reference. ProSolv is an excipient comprising a particulate agglomerate of coprocessed microcrystalline cellulose and from about 0.1% to about 20% silicon dioxide, by weight of the microcrystalline cellulose, the microcrystalline cellulose and silicon dioxide being in intimate association with each other, and the silicon dioxide portion of the agglomerate being derived from a silicon dioxide having a particle size from about 1 nanometer (nm) to about 100 microns (.mu.m), based on average primary particle size.

ProSolv® is free-flowing excipient that posses excellent disintegration properties, and importantly, improved compressibility relative to normal “off-the-shelf” commercially available microcrystalline cellulose when directly compressed. The advantages of ProSolv® are especially realized in pharmaceutical formulations prepared using wet granulation techniques. When utilized in wet granulation techniques, the ProSolv® provides a compressibility of normal “off-the-shelf” commercially available microcrystalline cellulose used in direct compression techniques. ProSolv® provides a compressibility which is substantially superior to the compressibility of normal “off-the-shelf” commercially available microcrystalline cellulose used in direct compression techniques.

The amount of silicified excipient present in the dosage forms described herein ranges from about 0.1% to about 50%, preferably from about 0.1% to about 20%.

In addition to the inclusion of a silicified excipient, the dosage forms may also contain an optional excipient such as, but not limited to, binder/fillers, disintegrants and superdisintegrants, lubricants, and antiadherents. Examples of suitable excipients include sucrose, dextrose, lactose, mannitol, starches, silicas, clays, microcrystalline cellulose, xylitol, fructose, sorbitol, disintegrants and superdisintergrants, such as crospovidone, croscarmellose and sodium starch glycolate, flavoring agents, acidifiers, sweeteners, taste-maskers, lubricants (e.g., magnesium stearate, stearic acid) and any combinations or mixtures of the foregoing.

Other suitable compression excipients for use in the dosage forms of the present invention may also pre-manufactured direct-compression excipients in place of part or all of the silicified excipients (e.g., ProSolv). Examples of such pre-manufactured direct compression excipients includ Emcocel® (microcrystalline cellulose, N.F.), Emdex® (dextrates, N.F.), and Tab-Fine® (a number of direct-compression sugars including sucrose, fructose, and dexrtose), all of which are commercially available from JRS Pharma Inc., Patterson, N.Y.). Other direct compression diluents include Anhydrous lactose (Lactose N.F., anhydrous direct tableting) from Sheffield Chemical, Union, N.J. 07083; Elcems® G-250 (Powered cellulose, N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltrodextrin) from Grain Processing Corp., Muscatine, Iowa 52761; Neosorb 60® (Sorbital, N.F., direct-compression) from Roquette Corp., 645 5th Ave., New York, N.Y. 10022; Nu-Tab® (Compressible sugar, N.F.) from Ingredient Technology, Inc., Pennsauken, N.J. 08110; Poly plasdone XL® (Crospovidone N.F., cross-linked polyvinylpyrrolidone) from ISP Corp, Wayne N.J. 07470; Primojel® (Sodium starch glycolate, N.F., carboxymethyl starch) from Generichem Corp., Little Falls, N.J. 07424; Spray-dried lactose® (Lactose N.F., spray dried) from Foremost Whey Products, Baraboo, Wis. 53913 and DMV Corp., Vehgel, Holland; and Sta-Rx 1500® (Starch 1500) (Pregelatinized starch, N.F., compressible) from Colorcon, Inc., West Point, Pa. 19486, calcium silicate (RxCIPIENTS FM1000) from Huber Materials, Germany. Pre-manufactured directed compression excipients may also comprise all or a portion of the inert diluent.

Rate of Release

Release properties of the active agent from the lipid coated substrate in the final dosage form may be altered depending on the lipid(s) chosen for a particular protective coating. The release properties of the active agent from the coated substrate in the final dosage form may further be altered by controlling the amount (thickness) of the lipid coating. The release properties may further be altered by the inclusion and amount of hydrophobic material incorporated into the protective coating. Any combination of the foregoing may be used together to achieve a desired release of active agent from the microencapsulated substrates.

For example, certain lipids, such as, but not limited to, monoglycerides, may provide a faster release of the active agent from the coated substrate. Other lipids, such as, but not limited to, triglycerides may provide a relatively slower, modified release of the active agent from the coated substrate. For purposes of the present invention, the term “modified release” is defined as a delayed release, a controlled release, a bi-phasic or multi-phasic release and pulsatiled release.

In addition to controlling the releasing rate by factors described herein, in embodiments of the invention where a modified release is desired, it is further contemplated that one or more modified release carriers may be incorporated into the dosage form. This may be accomplished, e.g., (i) by admixing one or more modified release carriers with a plurality of lipid coated active substrates;(ii) by applying a further coating comprising one or more modified release carriers onto the surface of the coated substrates (either with or without any additional admixed excipients); and (iii) any combination of the foregoing.

Suitable materials which may be included as the modified release carrier in such applications include hydrophilic and hydrophobic materials which are either pH-independent or pH-dependent in the environment of use (e.g., a dissolution media or in the gastro-intestinal tract when administered in-vivo). Such material include pharmaceutically acceptable polymers and copolymers, including cellulosics and acrylic and methacrylic acid polymers and copolymers, polysaccharides, gums, lipids (such as those set forth for use in encapsulation of the substrates in the invention), etc. This list is not meant to be exclusive. Examples of suitable materials include cellulose ethers and cellulose esters acrylic and methacrylic acid polymers and copolymers. Further specific examples of suitable modified release materials include alginates, xantham gum, guar gum, pectin, carageenan, gum Arabic, locust bean gum, carob gum, modified starch, methylhydroxyethylcellulose, carboxymethylcellulose, sodium carboxymethylcellulose, calcium carboxymethylcellulose, hydroxypropycellulose, hydroxypropylmethylcellulose, methyl hydroxyethylcellulose, ethylcellulose, poly(vinylpyrrolidone), polyacrylates, polylysines, poly (N-vinyl lactams), poly(ethylene oxide), poly (propylene oxide), polyacrylamides, polyacrylic acids, polyvinyl alcohols, polyvinyl ethers, polylactide, a polyglycolide, a poly (lactide-co-glycolide),a polyanhydride, a polyorthoester, polycaprolactones, polyphosphazenes, polysaccharides, proteinaceous polymers, soluble derivatives of polysaccharides, soluble derivatives of proteinaceous polymers, polypeptides, polyesters, polyorthoesters, poly-1,4-glucans (e.g., starch glycogen, amylose, amylopectin, and mixtures thereof), hydroxyalkyl derivatives of hydrolyzed amylopectin such as hydroxyethyl starch (HES), hydroxyethyl amylose, dialdehyde starch, methyl methacrylate, methyl methacrylate copolymers, ammonio methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly (methacrylic acid)(anhydride), polymethacrylate. polyacrylamide, poly(methacrylic acid anhydride), and glycidyl methacrylate copolymers, mixtures of any of the foregoing, and the like. Ammonio merhacrylate copolymers are well known in the art, and are described in NF XVII as fully polymerized copolymers of acrylic and methacrylic acid esters with a low content of quaternary ammonium groups. One commercially-available aqueous dispersion of ammoniomethacrylate copolymers is sold under the tradename Eudragit R (Rohm Pharma). One comercially-available aqueous dispersion of ethylcellulose is sold under the tradename Aquacoat® (FMC Corp.).

In certain embodiments, the orally disintegrating dosage forms of this invention, the active is taste masked and the release of the active agent has immediate release. In certain embodiments, the release of the active agent from the lipid coated active substrate does not have immediate release characteristics until it has been included in the dosage form. In the dosage form, the release is immediate whilst unexpectedly taste masking the active agent and/or providing stability.

### DETAILED DESCRIPTION OF THE PREFERRED EMBODIMENTS

The present invention will be firther appreciated in view of the following examples:

In the following examples soy or HSO is hydrogenated soybean oil, soy/EC is 90% hydrogenated soybean oil (Sterotex® HM NF, from Abitec) and 10% ethylcellulose (Ethylcellulose, Standard 4, from Dow Chemical). Castorwax/EC is 90% or 80% hydrogenated castor oil (HSO) (Casterwax® NF, from Caschem) and 10% or 20% ethylcellulose. ProSolv SMCC® 50, ProvSolv SMCC® 90, ProSolv SMCC® HD90 are all microcrystalline cellulose coprocessed with amorphous fumed silica, from JRS Pharma; Perlitol® is mannitol from Roquette; and Polyplasdone® XL is crospovidone from ISP Technologies; caffiene from Pharmline; acetaminophin (APAP)from Kangle; Ibuprofen from AnMar International Ltd.

### EXAMPLE I

### Process for Making Lipid Coated Active Substrates

As a representative example, acetaminophen was used as the active substrate. Twenty pounds (20 lbs) of acetaminophen was coated (microencapsuledated) using a solution of ethylcellulose in hydrogenated soybean oil (1.74 lbs), 1:9 ratio (EC/HSO). The solution, at 300° F. was top sprayed at a rate of about 0.34 lb/min onto a bed of acetaminophen in a modified fluid bed process. The bed temperature of about 115° F. was maintained, to achieve an even coating of the particles. A 92% load of the active agent was achieved. Other active substrates in the following examples were coated in a similar fashion. At 50% activity, 15 lbs of active substrate was coated with 15 lbs of coating.

The solution of lipid (for example hydrogenated soybean oil) and ethylcellulose were prepared by heating hydrogenated soybean oil (13.5 lbs) to a temperature of about 180° F. Ethylcellulose was added. By heating to 380° F., ethylcellulose (1.5 lbs) was then dissolved in the hydrogenated soybean oil to form a protective coating solution, at a 1:9 ratio.

Lipids that do not contain dissolved ethylcellulose are typically applied as a coating at lower temperatures, approx. 220° F.

### EXAMPLE II

### Orally Disintegrating Tablets Containing Soy Coated Acetaminophen

Soy coated acetaminophen at 92% activity was prepared as in Example I. The coated acetaminophen was mixed with excipients using a Turbula blender according to the following formula:
41 g coated APAP (92% active, soy coating)
40 g Pearlitol 200SD
14 g ProSolv SMCC90
3 g Polyplasdone
1 g Splenda
1 g Mg stearate

The powder blend was compressed into acceptable tablets on a K-International press. The tablet weight was about 505 mg, the hardness ranged from 0.8 to 1.1 kP. The acetaminophen in the orally disintegrating tablets were taste masked.

The tablets were tested for releases using a Dissolution Tester (Model VK 7000, Varian, Inc.) following USP 27/NF 22 with Apparatus 2. Dissolution vessels were filled with 900 ml pH 5.8 phosphate buffer at 37.0° C. The paddles were at 50 rpm. 5 ml samples were withdrawn from dissolution vessel at 30 min. Immediate release of the active was achieved with 96% release in 30 min.

### EXAMPLE III

### Orally Disintegrating Tablets Containing Soy/EC Coated Acetaminophen

Soy/EC coated acetaminophen at 92% activity was prepared as in Example I. The coated acetaminophen was mixed excipients using a Turbula blender according to the following formula:
110 g coated APAP (92% active)
50 g Pearlitol 200SD
30 g ProSolv 50
6 g Polyplasdone XL
2 g tartaric acid
1 g Mg-stearate

Tableting was done the same way as in the previous examples. The average weight of the tablets was 515 mg, the hardness range 0.6-0.8 kP. The tablets did not taste bitter. The tablets were tested for releases using a Dissolution Tester (Model VK 7000, Varian, Inc.) following USP 27/NF 22 with Apparatus 2. Dissolution vessels were filled with 900 ml pH 5.8 phosphate buffer at 37.0° C. The paddles were set at 50 rpm. 5 ml samples were withdrawn from dissolution vessel at 45 min. Immediate release of the active was achieved with 98% release in 45 min.

### EXAMPLE IV

### Orally Disintegrating Tablets Containing Soy Coated Ibuprofen

Soy coated ibuprofen at 80% activity was prepared as in Example I. The coated ibuprofen was mixed with excipients using a Turbula blender according to the following formula:
50 g coated ibuprofen (80% active)
50 g Peralitol 200SD
20 g ProSolv HD90
3 g Polyplasdone
1 g Splenda
1 g Mg-stearate

Pharmaceutically acceptable tablets were made on a K-International single-action tableting press. The tablet hardness (Dr. Schleusinger Tablet Tester 8M) ranged from 0.8-1.0 kP. The weight (Mettler) of a tablet was 500 mg on average. The tablets were found to be palatable, i.e., no bitterness or after-taste or throat-bite was noticable. A Varian VK7000 dissolution tester was used to measure the aqueous release of the active ingredient, following the USP #2 method. 900 ml pH 7.2 phosphate buffer was used as the medium. Based upon an average of two tablets, which resulted in 93% release after 60 minutes at 37° C.

### EXAMPLE V

### Orally Disintegrating Dosage Forms Containing the Coated Active Substrate, Caffeine (Soy/10% Ethylcellulose Coating)

Soy/EC coated caffeine at 70% activity was prepared as in Example I. Fifteen pounds of caffeine was coated with 6.4 lbs of HSO/EC, yielding particles with 70% activity. The lipid coated caffeine substrate was mixed with excipients using a Patterson-Kelly V-blender according to the following formula:
70 g coated caffeine (70% active, soy/EC coating)
70 g Pearlitol 200SD
20 g ProSolv 50
6 g Polyplasdone XL
2 g tartaric acid
2 g Ca-stearate

The powder blend was successfully compressed into tablets on a K-International press. Acceptable tablets were prepared; average weight: 730 mg; hardness range: 1.0-1.5 kP; no bitterness was detected.

### EXAMPLE VI

### Orally Disintegrating Dosage Forms Containing the Coated Active Substrate, Caffeine (Castorwax/10% Ethylcellulose Coating)

Castorwax/EC coated caffeine at 70% and 60% activities were prepared as in Example I. Each coated caffeine was mixed with excipients using a Patterson-Kelly V-blender according to the following formulas:
Formula I:
   70 g coated Caffeine (70% active)
   70 g Pearlitol 200SD
   20 g ProSolv 50
   6 g Polyplasdone XL
   2 g tartaric acid
   2 g Ca-stearate
Formula II:
   70 g coated Caffeine (60% active)
   70 g Pearlitol 200SD
   20 g ProSolv 50
   6 g Polyplasdone XL
   2 g tartaric acid
   2 g Ca-stearate

Blending and tableting were done the same way as in previous Examples. Acceptable tablets were prepared. The tablet weight was about 525 mg, the hardness ranged from 1.1 to 1.4 kP. The tablets were taste masked in each case.

The tablets were tested for releases using a Dissolution Tester (Model VK 7000, Varian, Inc.) following USP 27/NF 22 with Apparatus 2. Dissolution vessels were filled with 900 ml distilled water at 37.0° C. The paddles were set at 50 rpm. 5 ml samples were withdrawn from dissolution vessel at 60 min. Immediate release of the active achieved with 94.0% release for tablets made from 70% active caffeine microencapsulates and 76.4% release for caffeine tablet made from 60% active microencapsulates in 60 min.

### EXAMPLE VII

### Orally Disintegrating Dosage Forms Containing the Coated Active Substrate, Caffeine (Castorwax/20% Ethylcellulose Coating)

Castorwax/EC (80:20) coated caffeine at 60% activity was prepared as in Example I. The coated caffeine was mixed with excipients using a Petterson-Kelly V-blender according to the following formula:
70 g coated Caffeine (60% active)
70 g Pearlitol 200SD
20 g ProSolv 50
6 g Polyplasdone XL
2 g tartaric acid
2 g Ca-stearate

Blending and ableting were done the same way as in previous Examples. Acceptable tablets were prepared. The tablet weight was about 513 mg, the hardness ranged from 1.0 to 1.4 kP. The tablets were taste masked. The tablets were tested for releases using a Dissolution Tester (Model VK 7000, Varian, Inc.) as described in Example VI. Immediate release of the active was achieved with 84.1% release in 60 min.

### EXAMPLE VIII

### Sieving of Lipid Coated Substrate with Silicified Excipient

4.0 kg of soy-coated APAP (92% active)was bag mixed with 200 g CabOSil. The blend was sieved through a USSS 40-mesh screen by one person. After about an hour, the sieving completely stopped due to clogging. Weighing showed that only 2.3 kg of the 4.2 kg blend gad gone through the screen.

The test was repeated with ProSolv in place of CabOSil. Since silica constitutes only about 2% of ProSolv, a comparativly large amount of the latter was elected to be used. 2.0 kg of soy coated APAP (92% active) was bag mixed with 2.0 kg of ProSolv SMCC90. The blend was sieved through a USSS 40-mesh screen by the same person. The entire four-kg blend went through in about ten minutes, and there was no observable clogging on the screen.

### EXAMPLE IX

### Orally Disintegrating Tablets Containing Soy/EC Coated Acetaminophen

Acetaminophen (APAP) was coated with soy/EC to 92% active, as described in Example I. The following compression mix was made:

| Ingredient | Weight (kg) | Percent (% w/w) |
|---|---|---|
| Coated APAP | 14.0 | 33.7 |
| Pearlitol 200SD | 18.0 | 43.3 |
| Avicel PH102 | 3.0 | 7.2 |
| Starch 1500 | 3.0 | 7.2 |
| Polyplasdone | 2.2 | 5.3 |
| Orange | 0.46 | 1.1 |
| CabOSil | 0.60 | 1.4 |
| Mg-stearate | 0.30 | 0.7 |

To include the flow-aid CabOSil, the common practice is to co-sieve it with the other ingredients, preferably with the active ingredient. In this case, the co-sieving with the lipid coated active substrates proved to be difficult and extremely time-consuming, as in Example VIII. It took about three hours for three individuals, i.e., about nine man-hours to accomplish.

When CabOSil and Avicel were replaced with ProSolv SMCC 90, the ProSolv provided sufficient flow aid properties and the co-sieving time was reduced to 10 minutes. Once again, a similar result as in Example VIII.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specifications are accordingly to be regarded in an illustrative manner rather than a restrictive sense.

What is claimed is:

1. An orally disintegrating dosage form comprising:
a plurality of lipid coated active substrates, and a silicified excipient.

2. The orally disintegrating dosage form of claims 1, where the dosage form disintegrates in the oral cavity leaving swallowable residuals within tow minutes, without ingestion of water.

3. The orally disintegrating dosage forms claims 1 and 2, wherein the dosage form contains at least 10% silicified excipient by weight of total excipients.

4. The orally disintegrating dosage forms of claims 1-3, wherein the silicified excipient is a cellulose derivative.

5. The orally disintegrating dosage forms of claim 1-4, wherein the silicified excipient is silicified microcrystalline cellulose.

6. The orally disintegrating dosage forms of claim 5, wherein the silicified excipient is Prosolv®.

7. The orally disintegrating dosage form of claim 1, wherein the active concentration in the lipid coated substrate is at least 10%, preferably at least 40%, more preferably at least 65%, more preferably at least 75%, even more preferably at least 90%.

8. The orally disintegrating dosage form of claim 7, wherein the lipid is a wax or a fatty acid glycerol ester, and any combinations or mixtures thereof.

9. The orally disintegrating dosage form of claim 8, wherein the was is selected from a group consisting of paraffin wax, camauba wax, beeswax etc., and any combinations of mixtures thereof.

10. The orally disintegrating dosage form of claim 8, wherein the fatty acid glycerol ester selected from a group consisting of fully or partially hydrogenated monoglycerides, diglyceride and triglycerides, and any combinations or mixtures thereof.

11. The orally disintegrating dosage form of claim 10, wherein the fatty acid glycerol ester is a hydrogenated vegetable oil.

12. The orally disintegrating dosage form of claims 1-11, wherein the lipid coating comprises at least one additional acceptable additive selected from the group consisting of inert diluents, fillers, lubricants, binders, glidants, plasticizers, sensory masking agents, flavors, pH triggers, antioxidants, taste maskers, bitter blockers, and combinations thereof.

13. The orally disintegrating dosage form of claims 1-12, wherein the lipid coating on the substrate is at least 90% of the weight of the coated active substrate.

14. The orally disintegrating dosage form of claims 1-12, wherein the lipid coating on the substrate is at least 60% of the weight of the coated active substrate.

15. The orally disintegrating dosage form of claims 1-12, wherein the lipid coating on the substrate is at least 35% of the weight of the coated active substrate.

16. The orally disintegrating dosage form of claims 1-12, wherein the lipid coating on the substrate is at least 10% of the weight of the coated active substrate.

17. The orally disintegrating dosage form of claims 1-16, wherein the active is taste masked.

18. The orally disintegrating dosage form of claims 1-17, wherein the dosage form has an immediate release profile of the active.

19. The orally disintegrating dosage form of claims 1-17, wherein the dosage form has a modified release profile of the active.

20. The orally disintegrating dosage for of claim 19, wherein the modified release is a delayed-release.

21. The orally disintegrating dosage form of claim 19, wherein the modified release is a controlled-release.

22. The orally disintegrating dosage form of claim 19, wherein the active agent is a pulsatile release.

23. The orally disintegrating dosage form of claim 1-22, wherein the active agent is selected from the group consisting of antihistamines; antibiotics; antituberculosis agents; cholinergic agents; antimuscarinics; sympathomimetics; sympatholytic agents; miscellaneous autonomic drugs; iron preperations; haemostatics; cardiac drugs; antihypertensive agents; vasodilators; non-steroidal anti-inflammatory agents; opiate agonists; anticonvulsants; tranquilizers; chemotherapeutic agents; lipid lowering agents; H₂-antagonists; anti-coagulant and anti-platelet agents; bronchodilators; stimulants; barbiturates; sedatives; expectorants; antiemetics; gastro-intestinal drugs; antithyroid agents; genitourinary smooth muscle relaxants; vitamins; unclassified agents; steroids; glucocorticoids, and any combinations or mixtures of the foregoing.

24. The orally disintegrating dosage form of claim 23, wherein the active agent is acetaminophen.

25. The orally disintegrating dosage form of claims 23, wherein the active agent is guaifenesin.

26. The orally disintegrating dosage form of claims 23, wherein the active agent is ibuprofen.

27. The orally disintegrating dosage form claims 23, wherein the active agent is ranitidine.

28. The orally disintegrating dosage form of claims 23, wherein the active agent is caffeine.

29. The orally disintegrating dosage form of claims 24 and 26, wherein a portion of the active agent substrates comprise an opioid analgesic.

30. The orally disintegrating dosage form of claim 29, wherein the opioid active agent comprises hydrocodone or its salts or derivatives.

31. The orally disintegrating dosage form of claims 1-22, wherein the active agent is selected from the group consisting of vitamins, minerals, amino acids, herbal agents, botanical agents, enzymes, living or attenuated organisms such as prebiotics or probiotics and mixtures of any of the foregoing.

32 A method of preparing an orally disintegrating dosage form, comprising:
a) blending a plurality of lipid coated active agent substrates with excipients that include a silicified excipient;
b) optionally adding additional excipients;
c)tableting the pre-mix to form an orally disintegrating dosage form.

33. The method of preparing an orally disintegrating dosage form of claims 32, where the dosage form disintegrates in the oral cavity of any species leaving swallowable residuals within two minutes, without ingestion of water.

34. The method of preparing an orally disintegrating dosage form of claims 32 and 33, where in the dosage form contains at least 10% silicified excipient by weight of total excipients.

35. The method of preparing and orally disintegrating dosage form of claim 32-34, where in the silicified excipient is silicified microcrystalline cellulose.

36. The method of preparing an orally disintegrating dosage form of claim 32, where in the active concentration in the lipid coated substrate is at least 10%, preferably at least 40%, more preferably at least 65%, more preferably at least 75%, even more preferably at least 90%.

37. The method of preparing an orally disintegrating dosage form of claims 32-36, wherein the lipid coated active substrate comprises a lipid.

38. The method of preparing an orally disintegrating dosage form of claim 37, wherein the lipid is a wax or a fatty acid glycerol ester, and any combinations or mixtures thereof.

39. The method of preparing an orally disintegrating dosage form of claim 38, wherein the wax is selected from a group consisting of paraffin was, carnauba wax, paraffin wax, beeswax etc., and any combinations of mixtures thereof.

40. The method of preparing an orally disintegrating dosage form of claim 39, wherein the fatty acid glycerol ester selected from a group consisting of fully or partially hydrogenated monoglycerides, diglyceride and triglycerides, and any combinations or mixtures thereof.

41. The method of preparing an orally disintegrating dosage form of claim 40, wherein the fatty acid glycerol ester is a hydrogenated vegetable oil.

42. The method of preparing an orally disintegrating dosage form of claims 32-41, wherein the lipid coating comprises at least one additional acceptable additive.

43. The method of preparing an orally disintegrating dosage form of claim 42, wherein the additive is selected from the group consisting of inert diluents, fillers, lubricant, binders, glidants, plasticizers, sensory masking agents, flavors, pH triggers, antioxidants, taste maskers, bitter blockers, cellulose derivatives and combinations thereof.

44. The method of preparing an orally disintegrating dosage form of claim 43, wherein the cellulose derivative is ethylcellulose.

45. The method of preparing an orally disintegrating dosage form of claims 32-44, wherein the lipid coating on the substrate is lead than 90% of the weight of the coated active substrate.

46. The method of preparing an orally disintegrating dosage form of claims 32-44, wherein the lipid coating on the substrate is lead than 60% of the weight of the coated active substrate.

47. The method of preparing an orally disintegrating dosage form of claims 32-46, wherein the lipid coating on the substrate is less than 35% of the weight of the coated active substrate.

48. The method of preparing an orally disintegrating dosage form of claims 32-44, wherein the lipid coating on the substrate is less than 10% of the weight of the coated active substrate.

49. The method of preparing an orally disintegrating dosage form of claims 32-48, wherein the active is taste masked.

50. The method of preparing an orally disintegrating dosage form of claims 32-48, wherein the dosage form has an immediate release profile of the active substrate.

51. The method of preparing an orally disintegrating dosage form of claims 32-48, wherein the dosage form has a modified release profile of the active substrate.

52. The method of preparing an orally disintegrating dosage form of claim 51, wherein the modified release is a delayed-release.

53. The method of preparing an orally disintegrating dosage form of claim 51, wherein the modified release is a controlled-release.

54. The method of preparing an orally disintegrating dosage form of claim 51, wherein the modified release is a pulsatiled release.

55. The method of preparing an orally disintegrating dosage form of claims 32-54, wherein the active agent is selected from the group consisting of antihistamines; antibiotics; antituberculosis agents; cholinergic agents; antimuscarinics; sympathomimetics; sympatholytic agents; miscellaneous autonomic drugs; iron preperations; haemostatics; cardiac drugs; antihypertensive agents; vasodilators; non-steroidal anti-inflammatory agents; opiate agonists; anticonvulsants; tranquilizers; chemotherapeutic agents; lipid lowering agents; H₂-antagonists; anti-coagulant and anti-platelet agents; bronchodilators; stimulants; barbiturates; sedatives; expectorants; antiemetics; gastro-intestinal drugs; antithyroid agents; genitourinary smooth muscle relaxants; vitamins; unclassified agents; steroids; glucocorticoids, and any combinations or mixtures of the foregoing.

56. The method of preparing an orally disintegrating dosage form of claims 55, wherein the active agent is acetaminophen.

57. The method of preparing an orally disintegrating dosage form of claims 55, wherein the active agent is guaifenesin.

58. The method of preparing an orally disintegrating dosage form of claims 55, wherein the active agent is ibuprofen.

59. The method of preparing an orally disintegration dosage form of claims 55, wherein the active agent is ranitidine.

60. The method of preparing orally disintegrating dosage form of claims 55, wherein the active agent is caffeine.

61. The method of preparing an orally disintegrating dosage form of claims 56 and 58, wherein a portion of the active agent comprises an opioid analgesic.

62. The method of preparing an orally disintegrating dosage form of claim 61, wherein the opioid active agent comprises hydrocodone or its salts or derivatives.

63. The method of preparing an orally disintegrating dosage form of claims 32-54, wherein the active agent is selected from the group consisting of vitamins, minerals, amino acids, herbal agents, botanical agents, enzymes, living or attenuated organisms such as prebiotics or probiotics and mixtures of any of the foregoing.

64. An orally disintegrating dosage form, comprising
a plurality of substrates comprising an active agent microencapsulated with hydrogenated vegetable oil or an animal fat,
a particulate agglomerate of a co-processed microcrystalline cellulose and from about 0.1% to about 20% silicon dioxide, by weight of the microcrystalline cellulose, the microcrystalline cellulose and silicon dioxide being in intimate association with each other, and the silicon dioxide portion of the agglomerate being derived from a silicon dioxide having a particle size from about 1 nanometer (nm) to about 100 microns (.mu.m), based on average primary particle size,
the plurality of substrates and particulate agglomerate of coprocessed microcrystalline cellulose being directly compressed into a solid dosage form containing a therapeutically effective amount of the active agent and an effective amount of said coprocessed microcrystalline cellulose such that the dosage form disintegrates in the oral cavity of a human leaving swallowable residuals within two minutes, without ingestion of water.

65. An orally disintegrating dosage form, comprising
a compressed mixture of (1) a plurality of substrates comprising an active agent microencapsulated with hydrogenated vegetable oil or an animal fat and (2) a particulate agglomerate of coprocessed microcrystalline cellulose and from about 0.1% to about 20% silicon dioxide, by weight of the microcrystalline cellulose, the microcrystalline cellulose and silicon dioxide being in intimate association with each other, and the silicon dioxide portion of the agglomerate being derived from a silicon dioxide having a particle size from about 1 nanometer (nm) to about 100 microns (.mu.m), based on average primary particle size, the mixture of substrates and coprocessed microcrystalline cellulose being capable of being sieved through a USSS 40-mesh screen with no observable clogging on the screen.
